(19) United States
(12) Patent Application Publication
(10) Pub. No.: US 2014/0186423 A1
(43) Pub. Date: Jul. 3, 2014

(54) Transdermal Patch Having Ultrasound Transducer for Administering Thrombolytic Reagents to Patients Having a Protein Misfolding Disease

(71) Applicant: Mathew Gelfand, Lido Beach, NY (US)

(72) Inventor: Mathew Gelfand, Lido Beach, NY (US)

(21) Appl. No.: 14/140,609

(22) Filed: Dec. 26, 2013

Related U.S. Application Data
(63) Continuation-in-part of application No. 13/430,881, filed on Mar. 27, 2012.

(51) Int. Cl. A61K 9/70; A61K 38/17; A61K 38/49

(52) U.S. Cl. CPC A61K 9/7092; A61K 38/49; A61K 38/17
USPC 424/443; 424/94.64

(57) ABSTRACT

The present invention relates to a method and device for treating patients with a Protein Misfolding Disease by chronically administering an effective dose of a thrombolytic reagent and/or a thrombolytic reagent regulator over an intermittent period of time. The thrombolytic reagents degrade the misfolded proteins that accumulate and that can become toxic in such patients. The thrombolytic reagent regulators increase the catalytic efficiency of the thrombolytic reagents to reduce the amount of thrombolytic reagents necessary for an effective dose. The administration is administered transdermally via a transdermal patch that is equipped with an ultrasound transducer for enhancing the penetration of the thrombolytic reagent and thrombolytic reagent regulator into the bloodstream of a patient by increasing the permeability of the patient's skin.

This application is a continuation-in-part of U.S. application Ser. No. 13/430,881, filed on Mar. 27, 2012, which claims priority on U.S. Provisional Patent Application Ser. No. 61/558,650 filed Nov. 11, 2011, the disclosures of which are hereby incorporated by reference.

### FIELD OF THE INVENTION

The present invention relates to delivery systems and methods that provide for the chronic and intermittent transdermal administration of low doses of thrombolytic (fibrinolytic) reagents preferably in conjunction with at least one thrombolytic reagent regulator, such as but not limited to Annexin A2, over a prolonged period of time. The thrombolytic reagents may be tissue plasminogen activator (t-PA), streptokinase and/or urokinase and in conjunction with the thrombolytic reagent regulator, may be used for the treatment of patients suffering from one or more Protein Misfolding Diseases which include Alzheimer's disease, Atherosclerosis, Diabetes, Parkinson's disease, Mad Cow Disease and conditions of Multiple Sclerosis and Cystic Fibrosis. More specifically, the present invention relates to the transdermal administration of at least one of the fibrinolytic reagent regulator and at least one of the thrombolytic (fibrinolytic) reagents through a dermal patch where the dermal patch contains an ultrasound transducer to enhance the transdermal delivery of the thrombolytic (fibrinolytic) reagents.

### BACKGROUND OF THE INVENTION

### Tissue Plasminogen Activator

Thrombolytic (fibrinolytic) drugs act on the endogenous fibrinolytic system by converting plasminogen to the potent proteolytic enzyme plasmin. Plasmin in turn degrades fibrin clots and other plasma proteins. A number of thrombolytic (fibrinolytic) drugs, including urokinase, streptokinase and t-PA, are currently used to treat acute vascular disease.

Tissue plasminogen activator (t-PA) activates plasminogen to generate the proteinase plasmin which plays an important role in the degradation of fibrin. t-PA has been a particularly important pharmaceutical agent for use in treatment of vascular diseases due to its ability to dissolve blood clots in vivo. FIG. 3 provides a flow chart of the process for repairing an injury to a blood vessel. After a blood vessel injury occurs, a plasma protein called thromboplastin aids in blood coagulation by converting prothrombin to thrombin. Next, C-reactive protein (CRP) is stimulated by the tissue injury and in turn stimulates plasminogen activator inhibitor-1 (PAI-1) which stimulates t-PA. The t-PA activates plasminogen to generate plasmin which degrades the fibrin clot. Once the clot is dissolved, the extracellular matrix scaffold for tissue reconstruction begins the healing process. The foregoing description highlights the important connection that t-PA has throughout the whole blood vessel injury reconstruction process, including at the start of blood coagulation.

It has also been noted that t-PA degrades beta-amyloid plaques by activating plasminogen. (Wang et al., Beta-Amyloid Degradation and Alzheimer's Disease, J. Biomedicine and Biotech., 2006: 1-12 (2006)). t-PA was originally identified and purified from natural sources. Through the use of recombinant DNA techniques, DNA clones encoding the t-PA molecule have been identified and characterized leading to a determination of the DNA sequence and deduced amino acid sequence of t-PA (U.S. Pat. No. 4,853,330).

Several variants of t-PA have also been developed that address some of the disadvantages associated with the use of t-PA. These disadvantages include the short half life and fast clearance rate of t-PA. Such variants include those described in EPO Patent Publication No. 199,574, that have amino acid substitutions at the proteolytic cleavage sites at amino acid positions 275, 276 and 277. These forms are referred to as protease-resistant one-chain t-PA variants in that, unlike natural t-PA, they exist in either one chain or two chain form and are resistant to proteolytic cleavage. Such variants are thought to be superior to natural t-PA for pharmaceutical uses in that they are more stable. In addition, a variety of glycosylation mutants exist at positions 117, 119, 184-186 and 448-450 which exhibit higher specific activity than natural t-PA.

A general review of plasminogen activators and derivatives thereof can be found in Harris (1987, Protein Engineering 1:449-458); Pannekock et al. (1988, Fibrinolysis 2:123-132); and Ross et al. (1988, Annual Reports in Medicinal Chemistry, Vol. 23, Chapter 12), each of which is incorporated by reference herein.

Thrombolytic (fibrinolytic) drug therapy, and in particular, t-PA, has been used for treating acute ischemic stroke. There are two ways that t-PA has been administered—intravenously, or intra-arterially directly at the clot site. In these methods, the t-PA is delivered through the blood vessels to break up the clot that is disrupting the blood flow. Before a dose may be administered there are a number of criteria that first must be considered. First, there must be no evidence of bleeding in potential t-PA recipients because thrombolytic therapy can exacerbate a hemorrhagic stroke. Furthermore, patients taking blood thinners are also not allowed to receive t-PA. In addition, other important considerations include elevated blood pressure or blood sugar, recent surgery, low platelet count, and end-stage liver or kidney disorders. The foregoing therapies are can last for as long as 13 days but are recommended to not exceed a total t-PA administration of 40 mg to 90 mg over the life of the therapy because of the above considerations. As a result of the relatively short amount time of the foregoing therapies, i.e. a maximum therapy life of 13 days, the therapies are not able to sustain a desired level of a thrombolytic (fibrinolytic) reagent and/or fibrinogen and/or fibrin split products in the body of a patient over a prolonged period of time as in the present invention. The present invention, on the other hand, allows for the chronic administration of the thrombolytic (fibrinolytic) drugs over an intermittent schedule where the intermittent schedule can last for at least one month.

Annexin A2 is a cell-surface receptor for both plasminogen and its activator, t-PA, to convert plasminogen to plasmin very efficiently. Annexin A2 is a thrombolytic (fibrinolytic) and lipid regulator that stimulates thrombolytic reagents, such as t-PA, in strength by 10 to 60 times via a triple complex formation of t-PA-Annexin A2-plasminogen. The triple complex formation enables t-PA to convert plasminogen to clot-dissolving plasmin efficiently which is unlike the inefficient conversion when t-PA is alone.

FIG. 1 represents a flow chart that shows the known relationship between elevated cholesterol, blood cot formation and tissue plasminogen activator.

### Protein Misfolding Disease

Protein Misfolding Disease or proteopathy refers to a class of diseases in which certain proteins become structurally abnormal and as a result disrupt the function of cells, tissues and organs of the body. The proteins in this class of disease fail to fold into their normal configuration. In this misfolded state the proteins can become toxic or they can lose their normal function. Several neurodegenerative and other diseases are believed to result from the accumulation of amyloid fibrills formed by misfolded proteins. Diseases classified as Protein Misfolding Diseases include but are not limited to Alzheimer's disease, Parkinson's disease, Atherosclerosis, Diabetes mellitus, Mad Cow Disease, conditions of Multiple Sclerosis and Cystic Fibrosis, Primary systemic amyloidosis, Ig heavy-chain-associated amyloidosis, Secondary systemic amyloidosis, Senile systemic amyloidosis, Hemodialysis-related amyloidosis, Hereditary systemic ApoAI amyloidosis, Hereditary systemic ApoAII amyloidosis, Finnish hereditary amyloidosis, Hereditary lysozyme amyloidosis, Hereditary cystatin C amyloid angiopathy, Injection localized amyloidosis, Hereditary renal amyloidosis, Senile seminal vesicle amyloid, Familial subepithelial corneal amyloidosis, Cataract, Medullary thyroid carcinoma, Lewy-body dementia, Huntington's disease, Spongiform encephalopathies, Hereditary cerebral hemorrhage with amyloidosis, Amyotrophic lateral sclerosis, Familial British dementia, Familial Danish dementia, Familial amyloidotic polyneuropathy, Frontotemporal dementias, and Sickle cell anemia.

### Alzheimer's Disease

Alzheimer's disease is a type of Protein Misfolding Disease that causes problems with memory, thinking, and behavior. It is a progressive disease where symptoms gradually worsen over a number of years through the damaging and killing of brain nerve cells, i.e., neurons. Two suspected causes of the damaging and killing of neurons are plaques and tangles. Plaques are deposits of a protein fragment called beta-amyloid that build up in the spaces between nerve cells. The amyloid plaques are composed of a tangle of regularly ordered amyloid fibrillar aggregates that are characteristic of a Protein Misfolding Disease. Tangles are twisted fibers of another protein called tau that build up inside cells.

Most people develop plaques and tangles as they age; however, those diagnosed with Alzheimer's disease tend to develop substantially more. In addition, they also tend to develop in similar patterns, beginning in areas important for memory before spreading to other regions. It is not yet known what role plaques and tangles play in Alzheimer's disease, but it is believed that they play a role in blocking nerve cell communication and disrupting processes that cells need to survive. This leads to the damage and destruction of the neurons.

Patients diagnosed with Alzheimer's disease live an average of 8 years after their symptoms become noticeable but survival can range from 4 to 20 years. There is no current cure for Alzheimer's but treatments are administered that delay the progression of the disease.

The present invention is directed to a device and method for extinguishing the misfolded proteins that accumulate in patients with a type of Protein Misfolding Disease and specifically the beta-amyloid plaques that accumulate in a patient with Alzheimer's or other protein misfolding disease by the administration of a thrombolytic (fibrinolytic) reagent, such as t-PA, and a thrombolytic (fibrinolytic) reagent regulator, such as Annexin A2. In the present invention, the t-PA is administered intermittently at low doses over a long period of time to maintain serum concentrations between about 0.1 μg and 50 mgs of the complex formed between the thrombolytic reagent, such as but not limited to t-PA, and the thrombolytic reagent regulator, such as but not limited to Annexin A2. Alternatively, the thrombolytic reagent may be administered intermittently at low doses over a long period of time without a thrombolytic reagent regulator to maintain serum concentrations between about 0.1 mg and 50 mgs. The intermittent period may be every day, once a week, once a month, twice a week, twice a month, three times a week, three times a month, etc. The period of time that the therapy may last can be measured weekly and can be more than, less than or equal to a week, including two weeks, three weeks, four weeks, etc.; the period may be measured monthly and can be more than, less than, or equal to a month, including two months, three months, four months, five months, six months, etc.; or the period may be measured yearly and can be more than, less than or equal to a year, such as a year and a half, two years, two and a half years, three years, etc. In addition, the present invention is also directed to an anticoagulant therapy for mitigating the damaging effects that blood clots may have in patients with a type of Protein Misfolding Disease, particularly Alzheimer's disease.

### OBJECTS OF THE INVENTION

It is an object of the invention to provide an improved transdermal delivery system and method for treating patients afflicted with a Protein Misfolding Disease by chronically and intermittently administering a low dose of at least one thrombolytic (fibrinolytic) reagent regulator in conjunction with at least one thrombolytic (fibrinolytic) reagent.

It is another object of the invention to provide an improved transdermal delivery system and method that degrades the amyloid plaques and other misfolded proteins that form in patients suffering from a Protein Misfolding Disease.

It is yet another object of the invention to provide an improved transdermal delivery device that includes a transdermal patch that has an ultrasound transducer for providing enhanced delivery of one or more thrombolytic (fibrinolytic) reagent regulators and one or more thrombolytic (fibrinolytic) reagents to a Alzheimer's patient's bloodstream.

It is still another object of the invention to provide an improved delivery system of thrombolytic (fibrinolytic) reagent regulators and thrombolytic (fibrinolytic) reagents that extinguishes the beta-amyloid plaques that accumulate in the brain of a patient afflicted with Alzheimer's disease.

It is a further object of the invention to provide an improved transdermal delivery method that provides an enhanced administration of a thrombolytic (fibrinolytic) reagent regulator and a thrombolytic (fibrinolytic) reagent through the use of a transdermal patch that has an ultrasound transducer.

It is an even further object of the invention to provide a therapeutic method for treating patients with Alzheimer's disease or another Protein Misfolding disease by the intermittent administration of therapeutically effective doses of at least one thrombolytic (fibrinolytic) reagent regulator and at least one thrombolytic (fibrinolytic) reagent.

### SUMMARY OF THE INVENTION

The present invention relates to a method and apparatus for treating patients with a Protein Misfolding Disease, and in particular Alzheimer's disease, by the chronic and intermittent transdermal administration of low doses of at least one thrombolytic (fibrinolytic) reagent regulator and at least one thrombolytic (fibrinolytic) reagent such as tissue plasminogen activator (t-PA), streptokinase and/or urokinase. The transdermal administration is preferably via a dermal patch that is equipped with an enhancing agent, such as an ultrasound transducer; for increasing the permeability of the skin and thus the penetration of the reagents into a patient's bloodstream.

The thrombolytic (fibrinolytic) reagent in conjunction with the thrombolytic (fibrinolytic) reagent regulator should be administered in a way that is sufficient to achieve serum concentrations of between about 0.1 μg and 25 mgs of the complex formed by the reagent regulator and the reagent. Therefore, an object of the invention is to provide a dose-controlling transdermal applicator for thrombolytic (fibrinolytic) compositions and thrombolytic regulating compositions such as t-PA.

The present invention may be used to treat patients suffering from a Protein Misfolding Disease, such as Alzheimer's disease, Atherosclerosis, Diabetes mellitus, Parkinson's disease, Mad Cow Disease, and conditions of Multiple Sclerosis and Cystic Fibrosis by degrading the amyloid plaques and/or other misfolded proteins that have accumulated. Other protein misfolding diseases that the present invention may treat include but are not limited to Primary Systemic Amyloidosis, Ig heavy-chain-associated amyloidosis, Secondary systemic amyloidosis, Senile systemic amyloidosis, Hemodialysis-related amyloidosis, Hereditary systemic ApoAI amyloidosis, Hereditary systemic ApoAII amyloidosis, Finnish hereditary amyloidosis, Hereditary lysozyme amyloidosis, Hereditary cystatin C amyloid angiopathy, Injection localized amyloidosis, Hereditary renal amyloidosis, Senile seminal vesicle amyloid, Familial subepithelial corneal amyloidosis, Cataract, Medullary thyroid carcinoma, Lewy-body dementia, Huntington's disease, Spongiform encephalopathies; Hereditary cerebral hemorrhage with amyloidosis, Amyotrophic lateral sclerosis, Familial British dementia, Familial Danish dementia, Familial amyloidotic polyneuropathy, Frontotemporal dementias, Sickle cell anemia, Spinocerebral ataxia type 6, Fabry's disease, Spinobulbular muscular atrophy, Ataxia, Wilson disease, Atrial amyloidosis of heart, Dentatorubral pallidoluysian atrophy, Hereditary cerebral amyloid angiopathy, Gaucher's disease, Medullary carcinoma of thyroid, Cystic fibrosis, Marfan syndrome, Fragile X syndrome, Fragile XE syndrome, Alexander disease, Light chain amyloidosis, Fatak systemic amyloidosis, Machado-Joseph disease, Hereditary systemic amyloidosis, Myotonic dystrophy, Aquaporin-Vasopressin-1, Cancer, Prion disease, and Retinitis pigmentosa protein. The proteins associated with protein misfolding diseases include β-amyloid, Tau, Modified low-density lipoprotein, Islet amyloid polypeptide, Amylin, α-Synuclein, Immunoglobulin light chain, Immunoglobulin heavy chain, Serum amyloid, Transthyrein, β₂-Microglobulin, apolipoprotein A-I, apolipoprotein A-II, Gelsolin, Lysozyme, Cystatin G, Insulin, Fibrinogen, Lactoferrin, Seminogelin, Crystallin, Calcitonin, Huntington, Prion, Cystatin C, Superoxide dismutase, Abri, ADan, Hemoglobin, α_{1A}-voltage-dependent calcium channel subunit, α-Galactosidase A, Androgen receptor, Ataxins, ATP7B, Atrial natriuretic factor, Atrophin, β-Glucocerebrosidase, Cystic fibrosis transmembrane regulator protein, Fibrillin, Fragile X mental retardation-1 protein, Fragile X mental retardation-2 protein, Glial fibrillary acidic protein, Gonadotropin-release hormone receptor, Ig V_{L} domain, Machado-Joseph disease protein 1, Medin, Myotonic dystrophy protein kinase, Nephrogenic diabetes insipidus, p53, Rhodopsin and von Hippel Lindau protein.

The present invention, in particular, may be used to treat a person afflicted with Alzheimer's disease by degrading the beta-amyloid plaques that have formed in the patient's brain.

The delivery system of the present invention is preferably directed to a dermal patch that contains one or more thrombolytic (fibrinolytic) reagents, one or more thrombolytic (fibrinolytic) reagent regulators, and an ultrasound transducer to enhance the effects of the administration. The dermal patch is configured to provide the sustained release of the thrombolytic (fibrinolytic) reagent and thrombolytic (fibrinolytic) reagent regulator over a prolonged period of time during the administration. The thrombolytic (fibrinolytic) reagents and thrombolytic (fibrinolytic) reagent regulators may be a blended mixture that is located inside of the transdermal patch or the thrombolytic (fibrinolytic) reagents and thrombolytic (fibrinolytic) reagent regulators may be blended when they are being administered to a patient. The ultrasound transducer increases the permeability of the skin to allow for greater penetration of the reagents.

It is preferred that the dermal patch be applied to a patient to provide the sustained release of a therapeutic amount of the thrombolytic (fibrinolytic) reagents and thrombolytic (fibrinolytic) reagent regulators into a patient's bloodstream during an administration event. The administration event is preferably over a prolonged period of time and the sustained release is such that the serum concentration levels discussed herein are achieved. In another embodiment of the invention one or more thrombolytic (fibrinolytic) reagents and/or one or more thrombolytic (fibrinolytic) reagent regulators may be combined with slow release gel formulations which may be applied topically to the patient. The thrombolytic (fibrinolytic) reagents and thrombolytic (fibrinolytic) reagent regulators may be a blend such that the blend is contained in one gel mixture or separate gels that come into contact when they are being administered to a patient. In this embodiment an ultrasound may be applied to the slow release gel formulations to increase the permeability of the skin and allow for greater penetration of the reagent. In an alternate embodiment, the thrombolytic (fibrinolytic) reagents and/or thrombolytic (fibrinolytic) reagent regulators may be administered intravenously or in any other manner that is used in the art, such as but not limited to a microchip that may be implanted in a patient. In a further alternate embodiment, the thrombolytic (fibrinolytic) reagents and thrombolytic (fibrinolytic) reagent regulators may be administered by different means such as intravenously for the thrombolytic (fibrinolytic) reagents and transdermally for the thrombolytic (fibrinolytic) reagent regulators or vice versa.

The present invention may also be used to treat plaque that accumulates on the teeth of a patient. In this embodiment, the t-PA and Annexin A2 may be topically applied in a gel or other appropriate form to one or more teeth of a patient to eliminate and/or reduce the accumulation of the plaque. The t-PA and Annexin A2 in this embodiment may be placed on any surface and/or near any region of the teeth where plaque is accumulated or where it is desirable to prevent the accumulation of plaque. In particular, one region where the t-PA and Annexin A2 may be applied to the teeth is at or near the gum line of a patient. In this particular embodiment, the t-PA and Annexin A2 may be applied as such so that the complex it forms contacts the teeth and gums to ensure that the plaque is adequately reached by the t-PA and Annexin A2 complex, including plaque that accumulates below the gum line. By placing the t-PA and Annexin A2 so that it overlaps the teeth and gums, the t-PA and Annexin A2 complex can penetrate into the seam between the teeth and gums so that it reaches plaque below the gum line. The amount of t-PA and Annexin A2 that may be applied and the intermittency of the application may include that which has been disclosed herein for the transdermal application of t-PA and Annexin A2.

### BRIEF DESCRIPTION OF THE DRAWINGS

FIG. 1 is a flow chart that shows the relationship between cholesterol and blood clotting in a person's body.

FIG. 2 is a graphical representation of the relationship between the amount of t-PA to be administered and the frequency of t-PA administration.

FIG. 3 is a flow chart that shows the process for repairing an injury to a blood vessel.

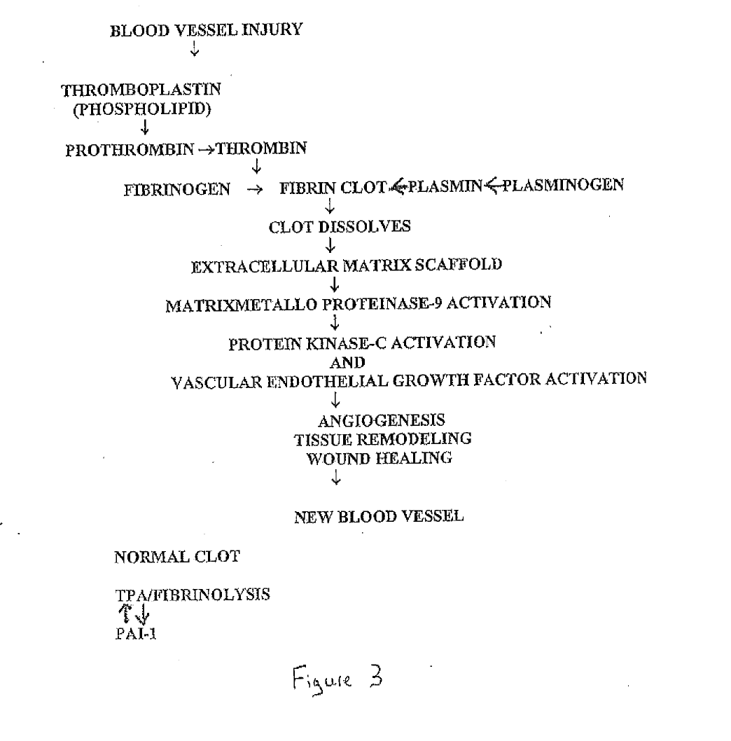

FIG. 4 is a graphical representation of the relationship between the amount of t-PA and Annexin A2 to be administered and the frequency of t-PA and Annexin A2 administration.

### DETAILED DESCRIPTION OF THE INVENTION

The present invention is directed to an improved delivery system and method for treating patients afflicted with a Protein Misfolding Disease, such as but not limited to Alzheimer's disease, Parkinson's disease, Atherosclerosis, Diabetes, Mad Cow Disease and conditions of Multiple Sclerosis and Cystic Fibrosis. More specifically, the invention relates to the chronic and intermittent transdermal administration of low doses of thrombolytic (fibrinolytic) reagent regulators, such as Annexin A2, and thrombolytic (fibrinolytic) reagents, such as t-PA, to degrade the misfolded proteins that have accumulated. In particular, the present invention can be used to treat patients suffering from Alzheimer's disease by degrading the beta-amyloid plaques that have accumulated in the brain.

The improved delivery system of the present invention is preferably a transdermal delivery system that delivers thrombolytic (fibrinolytic) reagents and reagent regulators, such as t-PA and Annexin A2, into the bloodstream of a patient with the aid of an enhancing agent. The transdermal drug delivery system is preferably a dermal patch that contains the t-PA and Annexin A2 to be administered to a human patient where the t-PA Annexin A2 are mixed with suitable carriers or excipient(s) at doses therapeutically effective to degrade amyloid plaques or prevent other vascular conditions. The dermal patch includes an enhancing agent in the form of an ultrasound transducer for increasing the pore size of the skin for greater penetration of the thrombolytic (fibrinolytic) reagent and reagent regulator into a patient's bloodstream.

The thrombolytic (fibrinolytic) reagents and reagent regulators may be administered daily, weekly, bi-weekly, monthly or yearly. The desired goal of any such delivery system is a constant long term delivery of thrombolytic (fibrinolytic) reagents and reagent regulators. Such thrombolytic (fibrinolytic) reagent regulators including but are not limited to Annexin A2 and such reagents include but are not limited to t-PA, streptokinase and urokinase.

### Thrombolytic (Fibrinolytic) Reagents

The thrombolytic (fibrinolytic) reagents to be used in the practice of the invention, herein defined as any reagents which have fibrinolytic activity, may be derived from a variety of different sources. For example, the t-PA may be produced in large quantities using recombinant DNA techniques well known to those skilled in the art such as those disclosed in U.S. Pat. No. 4,853,330 which is incorporated herein by reference. Alternatively, the t-PA may be obtained from a number of commercially available sources such as but not limited to Activase® and TNKase® supplied by Genentech, Inc. and isomers thereof.

When using t-PA, it is within the scope of the invention that variants of naturally occurring t-PA may also be used in the practice of the invention. In preferred embodiments, such variants of t-PA may have an increased half life or a slower rate of clearance from the body. For example, variants having amino acid substitutions at the proteolytic cleavage sites at position 275, 276 and 277 which render t-PA preparations more stable may be used. Glycosylation mutants at amino acids 117-119, 184-186 and 448-45 exhibit a higher specific activity and such variant may also be used in the practice of the invention, t-PA can also be modified to delete amino acids 51-87 which results in a variant having a slower clearance from plasma. These variants represent only a subset of the known variants of t-PA which may be used in the presently claimed delivery systems.

It is also within the scope of the present invention that thrombolytic (fibrinolytic) reagents other than t-PA may be used in the practice of the invention. Such agents include urokinase and streptokinase, both of which may be obtained from commercial sources (Urokinase, Abbott Laboratories; Streptokinase, Pharmacia Adria),

Method of Treating Patients with a Protein Misfolding Disease

The present invention relates to methods of treating patients with a Protein Misfolding Disease, such as but not limited to Alzheimer's disease, Parkinson's disease, Atherosclerosis, Diabetes, Mad Cow Disease and conditions of Multiple Sclerosis and Cystic Fibrosis, by the chronic and intermittent administration of low doses of thrombolytic (fibrinolytic) reagents and thrombolytic (fibrinolytic) reagent regulators. In particular, the complex formed by the present invention of the thrombolytic (fibrinolytic) reagents and thrombolytic (fibrinolytic) reagent regulators may be used to degrade amyloid plaques that accumulate in Alzheimer's patients and that can become toxic and result in a loss of brain function if not treated. Alternatively, the present invention may only include the administration of thrombolytic (fibrinolytic) reagents without the administration of a thrombolytic (fibrinolytic) reagent regulator.

Pharmaceutical compositions suitable for use in the present invention include compositions wherein the thrombolytic (fibrinolytic) reagent and reagent regulator ingredients are contained in an effective amount to achieve its intended purpose. More specifically, a therapeutically effective amount means an amount effective to degrade amyloid plaques or other misfolded proteins in the subject being treated. A therapeutically effective dose refers to that amount of the compound that results in plasma levels of the thrombolytic (fibrinolytic) reagent and reagent regulators which are sufficient to form a reagent and regulator complex that maintains the beneficial therapeutic effects.

The amount of composition administered will, of course, be dependent on the subject being treated, on the subject's weight, the severity of the affliction, the manner of administration and the judgment of the prescribing physician. It should be noted that the attending physician would know how to and when to terminate, interrupt or adjust therapy to lower dosage due to toxicity. Conversely, the attending physician would also know to adjust treatment to higher levels if the clinical response is not adequate (precluding toxicity).

In administering thrombolytic (fibrinolytic) reagents and reagent regulators to the patient, it is particularly important to monitor the patient for excessive bleeding or tendencies to bleed. A variety of different diagnostic tests, which are well known to those skilled in the art, may be used to access the patient's susceptibility to bleeding due to administration of the thrombolytic (fibrinolytic) reagents. Such assays include a complete blood count (CBC), or a determination of prothrombin or partial prothrombin time. It is preferred that a D-dimer test is performed before a dose of t-PA is administered. In the present invention, the thrombolytic (fibrinolytic) reagent regulators are administered to increase the catalytic efficiency of the thrombolytic (fibrinolytic) reagents by about 10 to about 60 times so that a reduced dose of thrombolytic (fibrinolytic) reagents can be administered with the same or greater effect than if a larger does of the thrombolytic (fibrinolytic) reagents were administered without reagent regulators. Administering this smaller dose of a thrombolytic reagent as a result of the reagent regulator further reduces the risk of excessive bleeding and other associated risk factors from occurring in a patient. This is especially important in the present invention's application to Alzheimer's disease where the majority of patients being treated are of older age and might have a tendency to suffer more sever consequences from any of the risk factors occurring that are associated with the administration of thrombolytic reagents.

The magnitude of a dose of the t-PA and Annexin A2 will vary with the patient to be treated. Again, it should be noted that the clinician or physician would know when to interrupt and/or adjust the treatment dose due to toxicity. The dose, and perhaps the dosage frequency, will also vary according to the age, body weight, and response of the individual patient.

In general, a total daily dose of t-PA and Annexin A2 should be sufficient to achieve serum concentration levels ranging between about 0.1 μg and 25 mgs of the t-PA-Annexin A2 complex. For smaller patients (less than 65 kg), a dose that is on the lower end of the aforementioned complex range should be administered. In addition, it is further recommended that patients over 65 years, and those with impaired renal, or hepatic function, initially receive low doses, and that they be titrated based on individual clinical response(s) and blood level(s).

### Thrombolytic (Fibrinolytic) Transdermal Drug Delivery System

The present invention also relates to a transdermal drug delivery system for transdermally delivering a quantity of thrombolytic (fibrinolytic) reagents, including t-PA, and reagent regulators, including Annexin A2, into the bloodstream of patients suffering from a Protein Misfolding Disease, such as but not limited to Alzheimer's disease, Parkinson's disease, Atherosclerosis, Diabetes, Mad Cow Disease and conditions of Multiple Sclerosis and Cystic Fibrosis. The transdermal drug delivery system is preferably a dermal patch that contains in the same compartment as a blended solution or in separate compartments the t-PA and Annexin A2 to be administered to a patient where the t-PA and Annexin A2 may be mixed separately or together with suitable carriers or excipient(s) at doses therapeutically effective to treat a patient with a Protein Misfolding Disease. The dermal patch contains an administration enhancing agent in the form of an ultrasound transducer for increasing the pore size of a patient's skin for greater penetration of the thrombolytic (fibrinolytic) reagent into the bloodstream.

The formulations of the present invention normally will consist of t-PA and Annexin A2 both or separately with a carrier, or diluted by a carrier. Some examples of the diluents or carriers which may be employed in the pharmaceutical compositions of the present invention are lactose, dextrose, sucrose, sorbitol, mannitol, propylene glycol, liquid paraffin, white soft paraffin, kaolin, microcrystalline cellulose, calcium silicate, silica polyvinylpyrrolidone, cetostearyl alcohol, starch, gum acacia, calcium phosphate, cocoa butter, oil of theobroma, arachis oil, alginates, tragacanth, gelatin, syrup B.P., methyl cellulose, polyoxyethylene sorbitan monolaurate, ethyl lactate and propylhydroxybenzoate, sorbitan trioleate, sorbitan sesquioleate and oleyl alcohol.

Because of the short shelf life of t-PA in solution, formulations of t-PA in aqueous solutions, gels, etc. are stored under refrigeration to preserve the activity of the t-PA. Lyophilized preparations of t-PA may be stored at room temperature and protected from excessive exposure to light without loss of activity.

The transdermal delivery of t-PA and Annexin A2 in accordance with the present invention can be designed so that the rate of delivery of the t-PA and Annexin A2 closely follows the rate of clearance of the t-PA-Annexin A2 complex from the patient's body, thus keeping constant levels of the t-PA-Annexin A2 complex in the blood, and thereby reducing t-PA and Annexin A2 waste and overdosing. The use of such a drug delivery system also provides a comfortable, convenient non-invasive method for unattended delivery of t-PA and Annexin A2 over a prolonged time period.

The transdermal delivery system is preferably in the form of a transdermal patch that is equipped with an ultrasound transducer. The patch preferably consists of an ultrasound transducer and a reservoir of drug material located behind a rate controlling membrane. The patch is impregnated with the t-PA and Annexin A2 in the same or separate compartments and placed on the skin of the patient. The ultrasound transducer may then be activated to increase skin permeability and allow the drug to penetrate readily into the body.

The transdermal patch is prepared to contain a solution of t-PA and Annexin A2. The patch may contain two separate solutions of t-PA and Annexin A2 or there may be one solution that is a blend of t-PA and Annexin A2. The t-PA and Annexin A2 may be dispersed in the solution, suspension or gel in a dissolved or undissolved state. The drug reservoir of the patch containing a solution, suspension or gel of t-PA and Annexin A2 also includes an ultrasound transducer to increase the skin's permeability. Other permeation enhancers may also be included in the dermal patch to increase the skin penetration of the t-PA and Annexin A2. Such permeation enhancers include those described in U.S. Pat. No. 4,573,966, which is incorporated by reference herein. Permeation enhancers may include plasticizer type enhancers such as lower alky and alkoxy esters of pharmaceutically acceptable fatty acids, fatty acid esters, fatty alcohols and similar hydrophobic compounds that are capable of increasing the permeability of drugs to the skin. In addition, solvent type enhancers may be used to increase the delivery of drugs through the skin. Such enhancers generally refer to relatively hydrophilic compounds having molecular weights of less than 200. More preferably, solvent type enhancers have a molecular weight of less than 150. They are also generally greater than 2 wt % soluble in water, and are preferably greater than 10 wt % soluble in water. Typically, solvent type enhancers include pharmaceutically acceptable lower alkyl alcohol, aryl alcohol, or polyol, for example, ethanol, propanol, butanol, benzyl alcohol, glycerin, or propylene glycol. as well as diluents, such as water or other additives. The solution of t-PA may be formulated to include vascular permeability factors (VPFs), as described in U.S. Pat. No. 5,503,843, which cause a rapid and reversible increase in blood vessel permeability. Such VPF may be added to the t-PA solution to facilitate the uptake of t-PA into the blood vessels of the skin. In addition, gelling agents may be added to increase the viscosity of the solution as is described in U.S. Pat. No. 5,503,843. The t-PA may also include diluents, stabilizers, biocides, antioxidants, anti-irritants and the like.

Because of the instability of t-PA in solution, it is desirable to design transdermal patches that can be stored at room temperature. Such a dermal patch may be designed, for example, with two compartments separated by a breakable barrier; one compartment contains lyophilized t-PA and Annexin A2 and the other compartment contains a solution or carrier, such as those described above, into which the t-PA and Annexin A2 are dissolved. Prior to the use of the patch, the barrier is broken, mixing the contents of both compartments thereby forming a drug reservoir containing a solution of t-PA and Annexin A2. Alternatively, a transdermal patch may be designed with a single breakable compartment containing lyophilized t-PA and Annexin A2, enclosed within the liquid carrier. Prior to use of the patch, the single compartment barrier is broken releasing the t-PA and Annexin A2 into the carrier solution. The patch is then placed in contact with the skin in such a way that the drug reservoir containing the t-PA and Annexin A2 solution is in contact with the skin.

The transdermal patch of the present invention may have a removable feature where a wearer can remove the patch even though the patch may still contain medicine to be administered. In this embodiment, the patch may have a resealing mechanism to re-seal the patch membrane to prevent any leakage of the medicine. The resealing mechanism may be in the form of a flap that may be resealed or it may be in the form of other ordinarily used resealing mechanisms. In addition, the patch adhesive for attaching the patch to the skin of a wearer may be adapted to re-adhere to a wearer's skin during reapplication of the patch to the skin. An example of a suitable adhesive is of the type used for Post-It brand sticky notes, but that are strong enough to form a secure adhesion with a patient's skin. Other adhesive mechanisms that are ordinarily used and that are within the spirit of the present invention may also be used. In a different embodiment, the patch may be designed to be removed once the reservoir of medicine has been depleted.

In addition, the use of the transdermal patch of the invention may be varied according to a physician's recommendations and a patient's needs. For example, the transdermal patch may be designed to chronically administer a dose for a specified period of time and the patch removed after the specified time period has expired. Alternatively, the dosage plan may require a new patch to be applied once the reservoir of the existing patch has been depleted to ensure a constant delivery of the thrombolytic (fibrinolytic) reagents and reagent regulators in a therapeutically effective amount.

### Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### Examples

### Example 1

### Transdermal Administration of Thrombolytic (Fibrinolytic) Reagents

The following example describes the administration of the thrombolytic (fibrinolytic) reagent t-PA utilizing a transdermal patch delivery system that contains an ultrasound transducer. The use of transdermal patches for the delivery of drugs through the skin is well known. Methods for the use of transdermal patches for delivery of drugs is described, for example, in the following U.S. Pat. Nos. 5,498,417, 5,503,844 and 5,503,843, each of which is incorporated by reference herein.

The following example illustrates the invention. It is not intended to limit the scope of the invention.

The t-PA (Activase, supplied by GENENTECH, Inc.) to be used in this example may be supplied in 50 mg vials. The vials should be reconstituted in either sterile water or a pharmaceutical composition compatible with use in a transdermal patch.

The transdermal patch is prepared to contain a solution of t-PA and an ultrasound transducer to boost the skin's penetration by the t-PA. The t-PA is dispersed in the solution, suspension or gel in a dissolved or undissolved state. The drug reservoir of the patch containing a solution, suspension or gel of t-PA also includes permeation enhancers in addition to the ultrasound transducer on the patch. Such permeation enhancers include those described in U.S. Pat. No. 4,573,966, which is incorporated by reference herein. Permeation enhancers may include plasticizer type enhancers such as lower alky and alkoxy esters of pharmaceutically acceptable fatty acids, fatty acid esters, fatty alcohols and similar hydrophobic compounds that are capable of increasing the permeability of drugs to the skin. In addition, solvent type enhancers may be used to increase the delivery of drugs through the skin. Such enhancers generally refer to relatively hydrophilic compounds having molecular weights of less than 200. More preferably, solvent type enhancers have a molecular weight of less than 150. They are also generally greater than 2 wt % soluble in water, and are preferably greater than 10 wt % soluble in water. Typically, solvent type enhancers include pharmaceutically acceptable lower alkyl alcohol, aryl alcohol, or polyol, for example, ethanol, propanol, butanol, benzyl alcohol, glycerin, or propylene glycol. as well as diluents, such as water or other additives. The solution of t-PA may be formulated to include vascular permeability factors (VPFs), as described in U.S. Pat. No. 5,503,843, which cause a rapid and reversible increase in blood vessel permeability. Such VPF may be added to the t-PA solution to facilitate the uptake of t-PA into the blood vessels of the skin.

The amount of t-PA contained in the patch is that amount necessary to deliver a therapeutically effective dose of t-PA. The treated patient's blood is monitored to determine the levels of circulating fibrinogen and/or fibrin split products. As the t-PA is administered, the patient should be monitored to prevent excessive bleeding which can result from the treatment with thrombolytic (fibrinolytic) reagents.

Once the transdermal patch has been prepared to contain an appropriate dose of t-PA, in a suitable solution, the patient's skin is overlaid with the transdermal patch. The patch is placed in contact with the skin in such a way that the side of the patch containing the t-PA solution side is in contact with the patient's skin and the ultrasound transducer is activated. The ultrasound transducer increases the pore size of the skin for greater penetration of the t-PA into the bloodstream,

### Example 2

### Predicted t-PA Dosage for a Once a Week Administration of t-PA

Example 2 is an example of a once a week administration of t-PA to a patient. Example 2 in no way represents a working example. It is predicted that 5 mg of t-PA will be administered to a patient a once a week. It is further predicted that the once a week administration can continue for at least one month.

### Example 3

### Predicted t-PA Dosage for a Twice a Week Administration of t-PA

Example 3 is an example of a twice a week administration of t-PA to a patient. Example 3 in no way represents a working example. It is predicted that 5 mg of t-PA will be administered to a patient twice a week. It is further predicted that the twice a week administration can continue for at least one month. It is preferred that a 5 mg dose of t-PA be administered at most twice a week.

### Relationship Between the Amount of Thrombolytic (Fibrinolytic) Reagents Administered and the Frequency of Administration

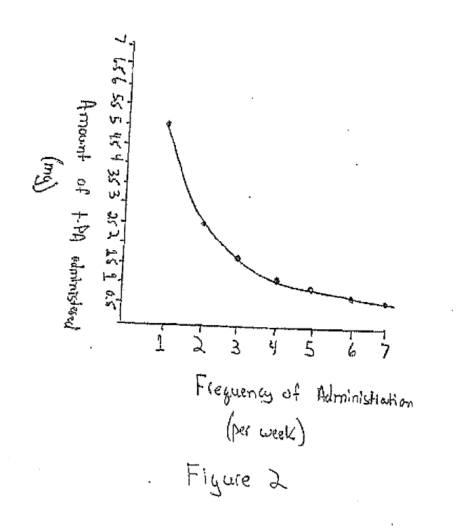

FIG. 2 is a graphical illustration of the effect that the frequency of t-PA administration will have on the amount of t-PA that is administered during each administration. The data in FIG. 2 is an estimate of therapeutically effective amounts of t-PA to be administered according to different administration frequencies and does not represent a working example. The frequency of administration in FIG. 2 varies from a weekly administration to a daily administration. FIG. 2 shows that an inverse relationship is predicted to occur between the frequency of administration and the dose of t-PA administered during each administration. It shows that as the frequency of administration is increased, that the dose of t-PA administered during each administration is predicted to decrease. The data estimated in FIG. 2 is the following: (1) for a once a week administration, about 5.0 mg of t-PA may be administered during each administration event; (2) for a twice a week administration, about 2.5 mg of t-PA may be administered; (3) for a three times a week administration, about 1.67 mg of t-PA may be administered; (4) for a four times a week administration, about 1.25 mg of t-PA may be administered; (5) for a five times a week administration, about 1.0 mg of t-PA may be administered; (6) for a six times a week administration, about 0.83 mg of t-PA may be administered; and (7) for a seven times a week administration, about 0.71 mg of t-PA may be administered.

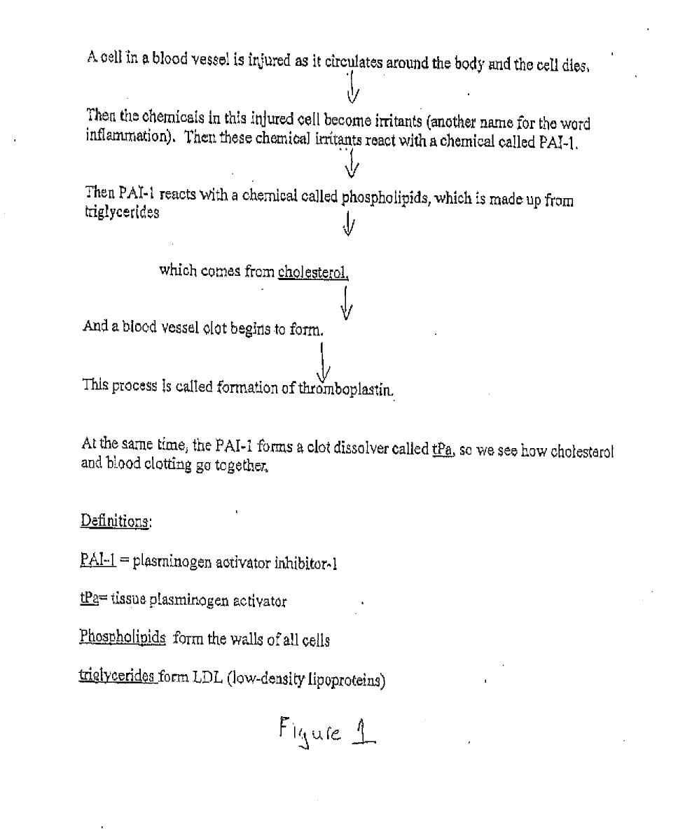

FIG. 2. shows the predicted relationship between the amount of t-PA to be administered and the frequency of administration of t-PA per week. However, the present invention also allows for the administration of t-PA on a monthly or yearly basis and the relationship between the amount of t-PA to be administered and the frequency of administration is predicted as being substantially the same as the predicted relationship that is shown in FIG. 1

It is predicted that therapeutically effective dosage amounts other than the amounts estimated in FIG. 2 can be used as long as the dosage is decreased as the frequency of administration is increased. A predicted range of a therapeutically effective dosage amount for a once a week administration can be from about 2.0 mg to about 10.0 mg of t-PA. A preferred predicted range would be from about 3.0 mg to about 9.0 mg of t-PA. A more preferred predicted range would be from about 4.0 mg to about 8.0 mg of a once a week t-PA administration. A most preferred predicted range would be from about 4.5 mg to about 7.0 mg of t-PA.

A predicted range of a therapeutically effective dosage amount for a twice a week administration of t-PA may be from about 1.0 mg to about 5.0 mg. Alternatively, the twice a week administration may be from about 1.5 mg to about 4.5 mg in a preferred predicted range. Even further, the twice a week administration may be from about 2.0 mg to about 4.0 mg in a more preferred predicted range. In a still further embodiment, the most preferred predicted range may be from about 2.0 mg to about 3.0 mg.

For a three times a week t-PA administration, the predicted range of a therapeutically effective dosage amount may be from about 0.7 mg to about 3.3 mg. A preferred predicted range may be from about 1.0 mg to about 3.0 mg. A more preferred predicted range may be from about 1.3 mg to about 2.7 mg. A most preferred predicted range may be from about 1.4 mg to about 2.4 mg.

A four times a week administration of t-PA may have a predicted range of a therapeutically effective dosage amount of from about 0.5 mg to about 2.5 mg. A preferred predicted range may be from about 0.75 mg to about 2.25 mg. A more preferred predicted t-PA range for a four times a week administration may be from about 1.0 mg to about 2.0 nag. A most preferred predicted range may be from about 1.0 mg to about 1.75 mg.

An administration of t-PA five times a week may have a predicted range of a therapeutically effective dosage amount of from about 0.4 mg to about 2.0 mg during each administration of t-PA. A five times a week t-PA administration preferred predicted range may be from about 0.6 mg to about 1.8 mg. A more preferred predicted range for a five times a week t-PA administration may be from about 0.8 mg to about 1.6 mg. A most preferred predicted range for t-PA administered five times a week may be from about 0.8 mg to about 1.4 mg.

t-PA being administered six times a week may have a predicted range of a therapeutically effective dosage amount of t-PA from about 0.3 mg to about 1.7 mg. A preferred predicted range of the amount of t-PA administered during an administration may be from about 0.45 mg to about 1.55 mg. A more preferred predicted range may be from about 0.60 mg to about 1.40 mg. A most preferred predicted range of the t-PA administered may be from about 0.60 mg to about 1.25 mg.

For a seven times a week t-PA administration schedule, a predicted range of a therapeutically effective amount of t-PA to be administered may be from about 0.25 mg to about 1.40 mg of t-PA. A preferred predicted range for this schedule may be from about 0.40 mg to about 1.25 mg. A more preferred predicted administration amount range for this administration schedule may be from about 0.55 mg to about 1.10 mg. A most preferred predicted range may be from about 0.55 mg to about 0.95 mg.

### Examples

### Example 4

### Transdermal Administration of Thrombolytic (Fibrinolytic) Reagents and Thrombolytic Reagent Regulators

The following example describes the administration of the thrombolytic (fibrinolytic) reagent t-PA and the thrombolytic (fibrinolytic) reagent regulator Annexin A2 utilizing a transdermal patch delivery system that contains an ultrasound transducer. The use of transdermal patches for the delivery of drugs through the skin is well known. Methods for the use of transdermal patches for delivery of drugs is described, for example, in the following U.S. Pat. Nos. 5,498,417, 5,503,844 and 5,503,843, each of which is incorporated by reference herein.

The following example illustrates the invention. It is not intended to limit the scope of the invention.

The t-PA (Activase, supplied by GENENTECH, Inc.) to be used in this example is supplied in 50 mg vials. The vials should be reconstituted in either sterile water or a pharmaceutical composition compatible with use in a transdermal patch.

The transdermal patch is prepared to contain a solution of t-PA, a separate solution of Annexin A2 and an ultrasound transducer to boost the skin's penetration by the t-PA. The t-PA is dispersed in the solution, suspension or gel in a dissolved or undissolved state. The drug reservoir of the patch containing a solution, suspension or gel of t-PA and Annexin A2 also includes permeation enhancers in addition to the ultrasound transducer on the patch. Such permeation enhancers include those described in U.S. Pat. No. 4,573,966, which is incorporated by reference herein. Permeation enhancers may include plasticizer type enhancers such as lower alky and alkoxy esters of pharmaceutically acceptable fatty acids, fatty acid esters, fatty alcohols and similar hydrophobic compounds that are capable of increasing the permeability of drugs to the skin. In addition, solvent type enhancers may be used to increase the delivery of drugs through the skin. Such enhancers generally refer to relatively hydrophilic compounds having molecular weights of less than 200. More preferably, solvent type enhancers have a molecular weight of less than 150. They are also generally greater than 2 wt % soluble in water, and are preferably greater than 10 wt % soluble in water. Typically, solvent type enhancers include pharmaceutically acceptable lower alkyl alcohol, aryl alcohol, or polyol, for example, ethanol, propanol, butanol, benzyl alcohol, glycerin, or propylene glycol. as well as diluents, such as water or other additives. The solution of t-PA may be formulated to include vascular permeability factors (VPFs), as described in U.S. Pat. No. 5,503,843, which cause a rapid and reversible increase in blood vessel permeability. Such VPF may be added to the t-PA solution to facilitate the uptake of t-PA and Annexin A2 into the blood vessels of the skin. In an alternate embodiment of the invention, the Annexin A2 and t-PA may be a pre-blended solution in a compartment in the transdermal patch.

The amount of t-PA and Annexin A2 contained in the patch is that amount necessary to deliver a therapeutically effective dose of the t-PA-Annexin A2 complex. The treated patient's blood is monitored to determine the levels of circulating fibrinogen and/or fibrin split products. As the t-PA-Annexin A2 complex is administered, the patient should be monitored to prevent excessive bleeding which can result from the treatment with thrombolytic (fibrinolytic) reagents.

Once the transdermal patch has been prepared to contain an appropriate dose of t-PA and Annexin A2, in a suitable solution, the patient's skin is overlaid with the transdermal patch. The patch is placed in contact with the skin in such a way that the side of the patch containing the t-PA-Annexin A2 solution side is in contact with the patient's skin and the ultrasound transducer is activated. The ultrasound transducer increases the pore size of the skin for greater penetration of the t-PA-Annexin A2 complex into the bloodstream.

### Example 5

### Predicted t-PA Dosage for a Once a Week Administration of t-PA and Annexin A2

Example 2 is an example of a once a week administration of t-PA and Annexin A2 to a patient. Example 2 in no way represents a working example. It is predicted that 2.5 mg of t-PA and 1.5 mg of Annexin A2 will be administered in separate dosage forms to a patient a once a week. It is further predicted that the once a week administration can continue for at least one month.

### Example 6

### Predicted t-PA Dosage for a Twice a Week Administration of t-PA and Annexin A2

Example 3 is an example of a twice a week administration of t-PA and Annexin A2 to a patient. Example 3 in no way represents a working example. It is predicted that 1.5 mg of t-PA and 1.0 mg of Annexin A2 will be administered to a patient twice a week. It is further predicted that the twice a week administration can continue for at least one month. It is preferred that a 1.5 mg dose of t-PA and a 1.5 mg dose of Annexin A2 be administered at most twice a week.

### Relationship Between the Amount of Thrombolytic (Fibrinolytic) Reagents and Reagent Regulators Administered and the Frequency of Administration

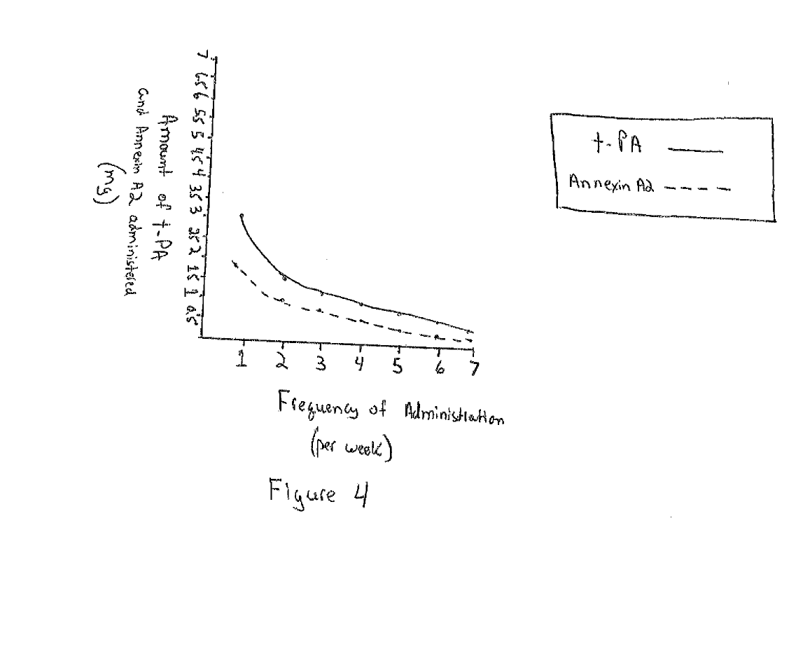

FIG. 4 is a graphical illustration of the effect that the frequency of t-PA and Annexin A2 administration will have on the amount of t-PA and Annexin A2 that is administered during each administration. The data in FIG. 4 is an estimate of therapeutically effective amounts of t-PA and Annexin A2 to be administered according to different administration frequencies and does not represent a working example. The frequency of administration in FIG. 4 varies from a weekly administration to a daily administration. FIG. 4 shows that an inverse relationship is predicted to occur between the frequency of administration and the dose of t-PA and Annexin A2 administered during each administration. It shows that as the frequency of administration is increased, the dose of t-PA and Annexin A2 administered during each administration is predicted to decrease. The data estimated in FIG. 4 is the following: (1) for a once a week administration, about 3.0 mg of t-PA and about 1.75 mg of Annexin A2 may be administered during each administration event; (2) for a twice a week administration, about 1.5 mg of t-PA and about 1.0 mg of Annexin A2 may be administered; (3) for a three times a week administration, about 1.25 mg of t-PA and about 0.90 mg of Annexin A2 may be administered; (4) for a four times a week administration, about 1.0 mg of t-PA and about 0.80 mg of Annexin A2 may be administered; (5) for a five times a week administration, about 0.75 mg of t-PA and about 0.50 mg of Annexin A2 may be administered; (6) for a six times a week administration, about 0.67 mg of t-PA and about 0.40 mg of Annexin A2 may be administered; and (7) for a seven times a week administration, about 0.50 mg of t-PA and about 0.33 mg of Annexin A2 may be administered.

FIG. 4 shows the predicted relationship between the amount of t-PA and Annexin A2 to be administered and the frequency of administration of t-PA and Annexin A2 per week. However, the present invention also allows for the administration of t-PA and Annexin A2 on a monthly or yearly basis and the relationship between the amount of t-PA and Annexin A2 to be administered and the frequency of administration is predicted as being substantially the same as the predicted relationship that is shown in FIG. 4.

It is predicted that therapeutically effective dosage amounts other than the amounts estimated in FIG. 4 can be used as long as the dosage is decreased as the frequency of administration is increased. A predicted range of a therapeutically effective dosage amount for a once a week administration can be from about 0.1 mg to about 7.5 mg of t-PA and from about 0.05 mg to about 5.0 mg of Annexin A2. A preferred predicted range would be from about 0.2 mg to about 6.0 mg of t-PA and from about 0.06 mg to about 4.0 mg of Annexin A2. A more preferred predicted range would be from about 0.5 mg to about 5.0 mg of a once a week t-PA and from about 0.08 mg to about 3.0 mg of a once a week Annexin A2 administration. A most preferred predicted range would be from about 0.75 mg to about 3.5 mg of t-PA and from about 0.1 mg to about 2.0 mg of Annexin A2.

A predicted range of a therapeutically effective dosage amount for a twice a week administration of t-PA and Annexin A2 may be from about 0.05 mg to about 4.0 mg of t-Pa and from about 0.025 mg to about 3.0 mg of Annexin A2. Alternatively, the twice a week administration may be from about 0.075 mg to about 3.0 mg of t-PA and from about 0.030 mg to about 2.5 mg of Annexin A2 in a preferred predicted range. Even further, the twice a week administration may be from about 0.1 mg to about 2.5 mg of t-Pa and from about 0.050 mg to about 1.75 mg of Annexin A2 in a more preferred predicted range. In a still further embodiment, the most preferred predicted range may be from about 0.25 mg to about 2.0 mg of t-PA and from about 0.075 mg to about 1.5 mg of Annexin A2.

For a three times a week t-PA and Annexin A2 administration, the predicted range of a therapeutically effective dosage amount may be from about 0.025 mg to about 3.0 mg of t-PA and from about 0.01 mg to about 2.0 mg of Annexin A2. A preferred predicted range may be from about 0.035 mg to about 2.5 mg of t-PA and from about 0.015 mg to about 1.75 mg of Annexin A2. A more preferred predicted range may be from about 0.045 mg to about 2.0 mg of t-Pa and from about 0.035 mg to about 1.5 mg of Annexin A2. A most preferred predicted range may be from about 0.1 mg to about 1.75 mg of t-PA and from about 0.055 mg to about 1.25 mg Annexin A2.

A four times a week administration of t-PA and Annexin A2 may have a predicted range of a therapeutically effective dosage amount of from about 0.01 mg to about 2.25 mg of t-PA and from about 0.0075 mg to about 1.75 mg of Annexin A2. A preferred predicted range may be from about 0.02 mg to about 2.0 mg of t-PA and from about 0.009 mg to about 1.5 mg of Annexin A2. A more preferred predicted t-PA and Annexin A2 range for a four times a week administration may be from about 0.025 mg to about 1.75 mg of t-PA and from about 0.01 mg to about 1.25 mg of Annexin A2. A most preferred predicted range may be from about 0.0.06 mg to about 1.5 mg of t-PA and from about 0.045 mg to about 1.0 mg of Annexin A2.

An administration of t-PA and Annexin A2 five times a week may have a predicted range of a therapeutically effective dosage amount of from about 0.005 mg to about 1.75 mg during each administration of t-PA and from about 0.004 mg to about 1.5 mg of Annexin A2. A five times a week t-PA and Annexin A2 administration preferred predicted range may be from about 0.01 mg to about 1.5 mg of t-PA and from about 0.006 to about 1.25 mg of Annexin A2. A more preferred predicted range for a five times a week t-PA and Annexin A2 administration may be from about 0.020 mg to about 1.25 mg of t-PA and from about 0.009 mg to about 1.0 mg of Annexin A2. A most preferred predicted range for t-PA and Annexin A2 administered five times a week may be from about 0.05 mg to about 1.0 mg of t-PA and from about 0.025 mg to about 0.75 mg of Annexin A2.

t-PA and Annexin A2 being administered six times a week may have a predicted range of a therapeutically effective dosage amount of t-PA from about 0.0035 mg to about 1.25 mg of t-PA and from about 0.002 mg to about 1.0 mg of Annexin A2. A preferred predicted range of the amount of t-PA and Annexin A2 administered during an administration may be from about 0.004 mg to about 1.0 mg of t-PA and from about 0.0025 to about 0.8 mg of Annexin A2. A more preferred predicted range may be from about 0.01 mg to about 0.8 mg of t-PA and from about 0.003 mg to about 0.6 mg of Annexin A2. A most preferred predicted range of the t-PA and Annexin A2 administered may be from about 0.035 mg to about 0.75 mg of t-PA and from about 0.009 mg to about 0.5 mg of Annexin A2.

For a seven times a week t-PA and Annexin A2 administration schedule, a predicted range of a therapeutically effective amount of t-PA and Annexin A2 to be administered may be from about 0.002 mg to about 1.0 mg of t-PA and from about 0.0015 mg to about 0.75 mg of Annexin A2. A preferred predicted range for this schedule may be from about 0.0025 mg to about 0.9 mg of t-PA and from about 0.002 mg to about 0.65 mg of Annexin A2. A more preferred predicted administration amount range for this administration schedule may be from about 0.003 mg to about 0.8 mg of t-Pa and from about 0.0025 mg to about 0.55 mg of t-PA. A most preferred predicted range may be from about 0.004 mg to about 0.7 mg of t-PA and from about 0.003 mg to about 0.45 mg of Annexin A2.

The administration schedules in the present invention are not limited to the schedules that are discussed above as the administration schedule of t-PA alone or t-PA and Annexin A2 may be once every two, three, four, five, or six or more weeks. In addition, the administration schedule may be twice every three weeks, five weeks, seven weeks, nine weeks, etc. Furthermore, the administration schedule may be calculated monthly or yearly rather than weekly and may include one administration every two months, three months, four months, five months, six months, etc. or an administration once a year, twice a year, three times a year, four times a year, five times a year, etc.

It is also important to note that the foregoing administration schedules may require the dosages to be administered equidistantly such that the time between each administration is substantially the same. In addition, the administration of the t-PA and Annexin A2 may have a constant rate of delivery regardless of the administration schedule. For example, the rate of delivery may be 0.5 mg/hr so that the administration period for the once a week administration is longer than the administration period for the twice a week administration since the once a week administration is a higher dose. Alternatively, the duration of the administration period for all of the different administration schedules may be substantially the same. In this embodiment, the rate of delivery of the doses will be adjusted so that the length of the administration period for a once a week administration substantially mirrors the length of the administration period for a different administration schedule.

The t-PA and Annexin A2 may be administered during an administration period on an hourly basis. For example, the period of administration may be one, two, three, four or more hours. The length of the administration period depends on the rate of t-PA and Annexin A2 delivery and the amount of t-PA and Annexin A2 to be delivered. The rate of t-PA and Annexin A2 administration may be the same or different. For example, a rate of delivery of t-PA and Annexin A2 of 0.5 mg/hr, will make a once a week administration of 3.0 mg of t-PA have an administration period of 6 hours and an administration of 1.75 mg of Annexin A2 to have an administration period of 3.5 hours. A range for a rate of delivery of t-PA and Annexin A2 in the present invention may be from about 0.1 mg/hr to about 2 mg/hr. A preferred rate of delivery range may be from about 0.2 mg/hr to about 1.5 mg/hr. A more preferred rate of delivery range may be from about 0.3 mg/hr to about 1.0 mg/hr. A most preferred rate of delivery range may be from about 0.4 mg/hr to about 0.6 mg/hr. It is to be noted that the present invention's administration is different from currently known drug therapies that use thrombolytic (fibrinolytic) drugs and thrombolytic (fibrinolytic) drug regulators for a variety of reasons. First, whereas currently known methods relate to the intravenous or intra-arterial administration of heavy doses of thrombolytic (fibrinolytic) drugs and thrombolytic (fibrinolytic) drug regulators, the present invention allows for a transdermal thrombolytic (fibrinolytic) drug and drug regulator administration. Second, currently used therapies of thrombolytic (fibrinolytic) drugs rely on a single heavy dosage of the thrombolytic (fibrinolytic) drug. The single heavy dosage prevents a thrombolytic (fibrinolytic) drug administration on a regular basis due to hemorrhagic considerations. The present invention, on the other hand, by intermittent administration and administration of a thrombolytic (fibrinolytic) drug regulator allows for smaller dosage administrations of thrombolytic (fibrinolytic) drugs which enables the drugs to be administered more frequently. And third, because the present invention can be administered on an intermittent basis, a sustained blood level of t-PA and Annexin A2 can be obtained during a therapy. Currently known and used thrombolytic (fibrinolytic) drug therapies, on the other hand, are not able to administer a heavy dosage on a regular basis and therefore are not capable of obtaining a sustained blood level.

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the claims.

What is claimed is:

1. A method for treating patients suffering from a protein misfolding disease comprising: the intermittent administration to a patient over a period of time of an effective dose of a thrombolytic reagent and a thrombolytic reagent regulator, wherein said thrombolytic reagent regulator reduces the amount of said thrombolytic reagent needed to create said effective dose than if said thrombolytic reagent was administered without said regulator, said thrombolytic reagent degrading the misfolded proteins that accumulate in patients having a protein misfolding disease, said treatment maintaining circulating blood levels of from about 0.1 mg to about 25 mg of said thrombolytic reagent in a patient, and said period of time of said intermittent administration being at least one month.

2. The method according to claim 1 wherein said thrombolytic reagent is tissue plasminogen activator (t-PA).

3. The method according to claim 2 wherein said thrombolytic reagent regulator is Annexin A2.

4. The method according to claim 3 wherein said intermittent administration is a once a week administration of t-PA and Annexin A2.

5. The method according to claim 4 wherein from about 1.0 mg to about 7.5 mg of t-PA and from about 0.5 to 5.0 mg of Annexin A2 is administered during each administration of t-PA and Annexin A2.

6. The method according to claim 5 wherein the rate of t-PA administration is from about 0.4 mg/hr to about 0.6 mg/hr.

7. The method according to claim 6 wherein the rate of Annexin A2 administration is from about 0.3 mg/hr to about 1.0 mg/hr.

8. The method according to claim 7 wherein the amount of t-PA administered is about 3.0 mg.

9. The method according to claim 8 wherein the amount of Annexin A2 administered is about 1.75 mg.

10. The method according to claim 9 wherein the rate of t-PA and Annexin A2 administration is 0.5 mg/hr.

11. The method according to claim 10 wherein each administration of t-PA and Annexin A2 lasts for about 10 hours.

12. The method according to claim 11 wherein the period of time is one year.

13. The method according to claim 12 wherein the period of time is six months.

14. The method according to claim 1 wherein the amount of time between each administration of said thrombolytic reagent and said thrombolytic reagent regulator over said period of time is substantially the same.

15. The method according to claim 2 wherein said protein misfolding disease is Alzheimer's disease.

16. The method according to claim 15 wherein said t-PA degrades beta-amyloid plaques that accumulate in the brain of a patient with Alzheimer's disease.

17. The method according to claim 1 wherein said thrombolytic reagent and said thrombolytic reagent regulator are administered transdermally.

18. The method according to claim 1 wherein a D-dimer test is performed before a thrombolytic reagent is administered to a patient.

19. A transdermal drug delivery system for delivering an effective dose of at least one thrombolytic reagent and at least one thrombolytic reagent regulator to a patient for the treatment of a protein misfolding disease, comprising: a transdermal patch having a rate controlling membrane with a reservoir of said thrombolytic reagent and said thrombolytic reagent regulator behind said rate controlling membrane and an ultrasound transducer for enhancing the penetration of said thrombolytic reagent into a patient's bloodstream by increasing the permeability of the patient's skin, said thrombolytic reagent regulator reducing the amount of said thrombolytic reagent that is needed to create an effective dose of said thrombolytic reagent.

20. The transdermal drug delivery system according to claim 19 wherein said thrombolytic reagent is t-PA and said thrombolytic reagent regulator is Annexin A2.

21. The transdermal drug delivery system according to claim 20 wherein the rate controlling membrane releases the t-PA and Annexin A2 at a rate of from about 0.1 mg/hr to about 2.0 mg/hr.

22. The transdermal drug delivery system according to claim 20 wherein the rate controlling membrane releases the t-PA and Annexin A2 at a rate of from about 0.4 mg/hr to about 0.6 mg/hr.

23. The transdermal drug delivery system according to claim 22 wherein the rate controlling membrane releases the t-PA at a rate of about 0.5 mg/hr.

24. A method for treating patients suffering from a protein misfolding disease comprising: intermittently administering from about 1.0 mg of tissue plasminogen activator (t-PA) to about 7.5 mg of t-PA and from about 0.5 to 5.0 mg of Annexin A2 to a patient, said t-PA t degrading the misfolded proteins that accumulate in the brain of patients having a protein misfolding disease, said Annexin A2 increasing the catalytic efficiency of said t-PA, and said treatment maintaining circulating blood levels of from about 0.1 mg to about 25 mg of said t-PA in a patient, wherein the intermittency of said intermittent administration is a once a week administration at a rate of administration of t-PA and Annexin A2 of 0.5 mg/hr, wherein each administration lasts for at least 10 hours, wherein the intermittent administration is continued for at least one month, and wherein a D-dimer test is performed before t-PA and Annexin A2 is administered.
